(19) United States
(12) Patent Application Publication
(10) Pub. No.: US 2009/0163475 A1
(43) Pub. Date: Jun. 25, 2009

(54) CRYSTALLINE FORM OF BENZAZEPINIUM MALEATE DERIVATIVE

(76) Inventors: Celine Bret, Kent (GB); Sarah Jane Burgess, Kent (GB); Simon John Hollas, Surrey (GB); Ian James King, Kent (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/915,393

(22) PCT Filed: May 22, 2006
(86) PCT No.: PCT/EP06/04954

§ 371 Date: Nov. 26, 2007

(30) Foreign Application Priority Data
May 24, 2005 (GB) 0510599.4

(51) Int. Cl. A61K 31/55; C07D 223/16; A61P 25/18

(52) U.S. Cl. 514/217.01; 540/594

(57) ABSTRACT

The present invention relates to a novel polymorphic form of 7-[4-(4-chlorobenzyloxy)benzenesulfonyl]-8-methoxy-3-methyl-2,3,4,5-tetrahydro-1H-3-benzazepinium maleate and a pharmaceutically acceptable solvate thereof, pharmaceutical formulation, process for its preparation and its use in medicine, in particular its use as an antipsychotic agent.

The present invention is concerned with a crystalline form of the maleate salt of 7-[4-(4-chlorobenzyloxy)benzenesulfonyl]-8-methoxy-3-methyl-2,3,4,5-tetrahydro-1H-3-benzazepine and its production and isolation.

7-[4-(4-Chlorobenzyloxy)benzenesulfonyl]-8-methoxy-3-methyl-2,3,4,5-tetrahydro-1H-3-benzazepine, as described and claimed in International Patent Application WO 03/099786 which is incorporated herein by reference, is described as being useful as antipsychotic agents for example in the treatment of schizophrenia, schizo-affective disorders and schizophreniform diseases and other disorders such as psychotic depression (which term includes bipolar depression, unipolar depression, single or recurrent major depressive episodes with or without psychotic features, catatonic features, melancholic features, atypical features or postpartum onset, seasonal affective disorder and dysthymia, depressive disorders resulting from a general medical condition including, but not limited to, myocardial infarction, diabetes, miscarriage or abortion), anxiety disorders (which includes generalised anxiety and social anxiety disorder), mania, acute mania, paranoid and delusional disorders.

The maleate salt of 7-[4-(4-chlorobenzyloxy)benzenesulfonyl)-8-methoxy-3-methyl-2,3,4,5-tetrahydro-1H-3-benzazepine is of particular importance since it enables 7-[4-(4-chlorobenzyloxy)benzenesulfonyl)-8-methoxy-3-methyl-2,3,4,5-tetrahydro-1H-3-benzazepine to be conveniently formulated in, for example, tablets for oral administration. There is thus the need to produce 7-[4-(4-chlorobenzyloxy)benzenesulfonyl)-8-methoxy-3-methyl-2,3,4,5-tetrahydro-1H-3-benzazepinium maleate in as pure and as highly crystalline a condition as possible in order to fulfil exacting pharmaceutical requirements and specifications.

The process by which 7-[4-(4-chlorobenzyloxy)benzenesulfonyl)-8-methoxy-3-methyl-2,3,4,5-tetrahydro-1H-3-benzazepinium maleate is produced also needs to be one which is convenient to operate on a plant scale.

7-[4-(4-Chlorobenzyloxy)benzenesulfonyl)-8-methoxy-3-methyl-2,3,4,5-tetrahydro-1H-3-benzazepinium maleate may be prepared, for example, as described in International Patent Application WO 05/051916, by contacting appropriate stoichiometric amounts of 7-[4-(4-chlorobenzyloxy)benzenesulfonyl]-8-methoxy-3-methyl-2,3,4,5-tetrahydro-1H-3-benzazepine free base with maleic acid in a suitable solvent. The free base of 7-[4-(4-chlorobenzyloxy)benzenesulfonyl]-8-methoxy-3-methyl-2,3,4,5-tetrahydro-1H-3-benzazepine may, for example, be in solution with the appropriate acid added as a solid or both the free base of 7-[4-(4-chlorobenzyloxy)benzenesulfonyl]-8-methoxy-3-methyl-2,3,4,5-tetrahydro-1H-3-benzazepine and the appropriate acid may independently be in solution. The crystalline form of 7-[4-(4-chlorobenzyloxy)benzenesulfonyl)-8-methoxy-3-methyl-2,3,4,5-tetrahydro-1H-3-benzazepinium maleate reported in Example 1 of WO 05/051916 with the characterising data included therein, is a specific polymorphic form hereinafter designated Anhydrate Form 1.

It has now been found that 7-[4-(4-chlorobenzyloxy)benzenesulfonyl)-8-methoxy-3-methyl-2,3,4,5-tetrahydro-1H-3-benzazepinium maleate can be prepared in a new crystalline form, hereinafter designated Form 2, for which the manufacturing process for the said new crystalline form fulfils the desirable features described above.

Suitable solvents for solubilising 7-[4-(4-chlorobenzyloxy)-benzenesulfonyl]-8-methoxy-3-methyl-2,3,4,5-tetrahydro-1H-3-benzazepine free base include for example alcohols such as ethanol and methanol, ketones such as acetone, halogenated hydrocarbons such as dichloromethane, and ethers such as tetrahydrofuran. If the maleic acid is to be added as a solution in a solvent, the solvent used may include acetone, ethanol, methanol, propan-2-ol or water.

For the preparation of the Form 2 maleate salt, the concentration of 7-[4-(4-chlorobenzyloxy)benzenesulfonyl]-8-methoxy-3-methyl-2,3,4,5-tetrahydro-1H-3-benzazepine free base may be for example in the range 3 to 25% weight/volume. The concentration of maleic acid when used in solution may be for example in the range 0.5 to 5 molar. Elevated temperatures (for example up to the boiling point of the solvent used) may be used to increase the solubility of the free base and/or the acid.

Crystalline maleate Form 2 salt may be prepared by directly crystallising from a solvent in which the salt has limited solubility, or by triturating or otherwise crystallising a non-crystalline salt. For example, 7-[4-(4-chlorobenzyloxy)benzenesulfonyl]-8-methoxy-3-methyl-2,3,4,5-tetrahydro-1H-3-benzazepinium maleate may be recrystallised from a variety of organic solvents, such as acetone, acetonitrile, butanone, 1-butanol, ethanol, methanol, 1-propanol or tetrahydrofuran or mixtures of such solvents. The mixtures of such solvents may additionally include water so that aqueous mixtures of the aforementioned solvents may also be used for the recrystallisation.

An improved yield of the salts may be obtained by the evaporation of some or all of the solvent or by crystallisation at elevated temperature followed by controlled cooling, for example in stages. Careful control of the precipitation temperature and seeding may be used to improve the reproducibility of the production process and the particle size distribution and form of the product. Individual polymorphs may be for example crystallized directly from a solution of the salt, although recrystallizing a solution of a particular polymorph using seeds of that polymorph may also be carried out.

The Form 2 maleate salt of 7-[4-(4-chlorobenzyloxy)benzenesulfonyl)-8-methoxy-3-methyl-2,3,4,5-tetrahydro-1H-3-benzazepine can exist as a solvate for example as a hydrate, or in an unsolvated form, for example an anhydrate.

The present invention thus provides 7-[4-(4-chlorobenzyloxy)benzenesulfonyl)-8-methoxy-3-methyl-2,3,4,5-tetrahydro-1H-3-benzazepinium maleate in a new crystalline form, designated Form 2. Therefore, as a first aspect of the invention there is provided one or more chemical entities selected from a crystalline form of 7-[4-(4-chlorobenzyloxy)benzenesulfonyl]-8-methoxy-3-methyl-2,3,4,5-tetrahydro-1H-3-benzazepinium maleate, Form 2 and a pharmaceutically acceptable solvate thereof.

Depending on the solvent from which the Form 2 maleate is recovered, the Form 2 maleate may be obtained as a solvate and such a solvate also forms one aspect of the present invention. The solvate may be a pharmaceutically acceptable solvate. Suitable solvates include acetone, acetonitrile, 2-butanone, cyclohexanone, dioxan, N,N-dimethylformamide, ethyl acetate, tetrahydrofuran, toluene and water; or mixtures thereof.

Alternatively, the Form 2 maleate may be obtained as an anhydrate. The anhydrate may contain less than 2% water, for example less than 1% water. The Form 2 maleate anhydrate demonstrates particular stability with respect to hygroscopicity and loss of water. Furthermore, the Form 2 maleate anhydrate demonstrates reversible changes when exposed to very high humidity.

Therefore, in a further aspect there is provided one or more chemical entities selected from the Form 2 maleate and a pharmaceutically acceptable solvate thereof in isolated form. In a yet further aspect there is provided one or more chemical entities selected from the Form 2 maleate and a pharmaceutically acceptable solvate thereof which is substantially free of alternative salts, alternative solvates, or free base of 7-[4-(4-chlorobenzyloxy)benzenesulfonyl)-8-methoxy-3-methyl-2,3,4,5-tetrahydro-1H-3-benzazepinium maleate or other impurity.

By “substantially free of alternative salts, alternative solvates or free base of 7-[4-(4-chlorobenzyloxy)benzenesulfonyl)-8-methoxy-3-methyl-2,3,4,5-tetrahydro-1H-3-benzazepinium maleate or other impurity” is meant containing less than 10%, for example less than 5%, such as less than 2%, of alternative salts, alternative solvates or free base of 7-[4-(4-chlorobenzyloxy)benzenesulfonyl)-8-methoxy-3-methyl-2,3,4,5-tetrahydro-1H-3-benzazepinium maleate or other impurity. The term “other impurity” includes any compound other than 7-[4-(4-chlorobenzyloxy)benzenesulfonyl)-8-methoxy-3-methyl-2,3,4,5-tetrahydro-1H-3-benzazepinium maleate.

A further aspect of the invention provides 7-[4-(4-chlorobenzyloxy)benzenesulfonyl)-8-methoxy-3-methyl-2,3,4,5-tetrahydro-1H-3-benzazepinium maleate, Anhydrate Form 2, characterised by its X-ray powder diffraction pattern as shown in FIG. 1 and/or by its Raman spectrum as shown in FIG. 2.

Accordingly, a further aspect of the present invention provides 7-[4-(4-chlorobenzyloxy)benzenesulfonyl]-8-methoxy-3-methyl-2,3,4,5-tetrahydro-1H-3-benzazepinium maleate, Anhydrate Form 2 characterised by its X-Ray powder diffraction (XRPD) pattern with signals substantially as listed in Table 1.

A further aspect of the invention provides 7-[4-(4-chlorobenzyloxy)benzenesulfonyl)-8-methoxy-3-methyl-2,3,4,5-tetrahydro-1H-3-benzazepinium maleate, Form 2, tetrahydrofuran solvate, characterised by its X-ray powder diffraction pattern as shown in FIG. 4 and/or by its Raman spectrum as shown in FIG. 5.

A further aspect of the present invention provides 7-[4-(4-chlorobenzyloxy)benzenesulfonyl]-8-methoxy-3-methyl-2,3,4,5-tetrahydro-1H-3-benzazepinium maleate, Form 2, tetrahydrofuran solvate characterised by its X-Ray powder diffraction (XRPD) pattern with signals substantially as listed in Table 2.

Solvates of the Form 2 maleate salt may be prepared by conventional means from a solution of the Form 2 maleate salt, or alternatively from a solution of the maleate salt prepared according to description 1 of the present invention. For example, the tetrahydrofuran solvate of the Form 2 maleate salt may be prepared by recrystallisation from tetrahydrofuran after seeding with Form 2 maleate at elevated temperature, for example, about 50° C. and subsequent cooling of the resultant mixture to room temperature until crystallisation occurs.

As used herein, the phrase “the Form 2 maleate salt and a pharmaceutically acceptable solvate thereof” is intended to include either the Form 2 maleate salt, a pharmaceutically acceptable solvate of the Form 2 maleate salt, or mixtures of the Form 2 maleate salt and one or more pharmaceutically acceptable solvates. It will be understood by the person skilled in the art that the amount of solvent in any particular solvate may vary and that the term “pharmaceutically acceptable solvate” is intended to cover solvates with varying amounts of solvent present.

### DESCRIPTION OF FIGURES

FIG. 1 shows X-Ray powder diffraction (XRPD) data obtained for 7-[4-(4-chlorobenzyloxy)benzenesulfonyl)-8-methoxy-3-methyl-2,3,4,5-tetrahydro-1H-3-benzazepinium maleate, Anhydrate Form 2 prepared as described in Example 2.

7-[4-(4-Chlorobenzyloxy)benzenesulfonyl)-8-methoxy-3-methyl-2,3,4,5-tetrahydro-1H-3-benzazepinium maleate, Anhydrate Form 2 as described in Example 2 is characterised by having an XRPD pattern with signals substantially as listed in Table 1.

FIG. 2 shows the Raman spectrum of 7-[4-(4-chlorobenzyloxy)benzenesulfonyl]-8-methoxy-3-methyl-2,3,4,5-tetrahydro-1H-3-benzazepinium maleate, Anhydrate Form 2 prepared as described in Example 2.

FIG. 3 shows a Differential Scanning Calorimetry (DSC) thermogram of 7-[4-(4-chlorobenzyloxy)benzenesulfonyl]-8-methoxy-3-methyl-2,3,4,5-tetrahydro-1H-3-benzazepinium maleate, Anhydrate Form 2 prepared as described in Example 2.

FIG. 4 shows X-Ray powder diffraction (XRPD) data obtained for 7-[4-(4-chlorobenzyloxy)benzenesulfonyl)-8-methoxy-3-methyl-2,3,4,5-tetrahydro-1H-3-benzazepinium maleate, Form 2, tetrahydrofuran solvate prepared as described in Example 3.

7-[4-(4-Chlorobenzyloxy)benzenesulfonyl)-8-methoxy-3-methyl-2,3,4,5-tetrahydro-1H-3-benzazepinium maleate, Form 2, tetrahydrofuran solvate as described in Example 3 is characterised by having an XRPD pattern with signals substantially as listed in Table 2.

FIG. 5 shows the Raman spectrum of 7-[4-(4-chlorobenzyloxy)benzenesulfonyl]-8-methoxy-3-methyl-2,3,4,5-tetrahydro-1H-3-benzazepinium maleate, Form 2, tetrahydrofuran solvate prepared as described in Example 3.

FIG. 6 shows a Differential Scanning Calorimetry (DSC) thermogram of 7-[4-(4-chlorobenzyloxy)benzenesulfonyl]-8-methoxy-3-methyl-2,3,4,5-tetrahydro-1H-3-benzazepinium maleate, Form 2, tetrahydrofuran solvate prepared as described in Example 3.

It will be recognised that spectra and diffraction data will vary slightly according to various factors such as the temperature, concentration and instrumentation used. The skilled person will recognise that XRPD peak positions are affected by differences in sample height. The peak positions quoted herein are thus subject to a variation of +/−0.15 degrees 2-theta.

7-[4-(4-Chlorobenzyloxy)benzenesulfonyl]-8-methoxy-3-methyl-2,3,4,5-tetrahydro-1H-3-benzazepinium maleate, Form 2 salt and pharmaceutically acceptable solvates thereof have been found to exhibit affinity for dopamine receptors, in particular the D₃ and D₂ receptors, and are useful in the treatment of disease states which require modulation of such receptors, such as psychotic conditions. These salts have also been found to have greater affinity for dopamine D₃ than for D₂ receptors. The therapeutic effect of currently available antipsychotic agents (neuroleptics) is generally believed to be exerted via blockade of D₂ receptors; however this mechanism is also thought to be responsible for undesirable extrapyramidal side effects (eps) associated with many neuroleptic agents. Without wishing to be bound by theory, it has been suggested that blockade of the dopamine D₃ receptor may give rise to beneficial antipsychotic activity without significant eps (see for example Sokoloff et al, Nature, 1990; 347: 146-151; and Schwartz et al, Clinical Neuropharmacology, Vol 16, No. 4, 295-314, 1993).

7-[4-(4-Chlorobenzyloxy)benzenesulfonyl]-8-methoxy-3-methyl-2,3,4,5-tetrahydro-1H-3-benzazepinium maleate, Form 2 salt and pharmaceutically acceptable solvates thereof have also been found to have antagonist affinity for the serotonin 5-HT_{2C}, 5-HT_{2A} and 5-HT₆ receptors. These properties may give rise to anti-psychotic activity (e.g. improved effects on cognitive dysfunction) activity with reduced eps, and/or anxiolytic/antidepressant activity. These could include, but are not limited to, attenuation of cognitive symptoms via 5-HT₆ receptor blockade (see Reavill, C. and Rogers, D. C., 2001, Investigational Drugs 2, 104-109), and reduced anxiety (see for example Kennett et al., Neuropharmacology 1997 April-May; 36 (4-5): 609-20), protection against eps (Reavill et al., Brit. J. Pharmacol., 1999; 126: 572-574) and antidepressant activity (Bristow et al., Neuropharmacology 39:2000; 1222-1236) via 5-HT_{2C} receptor blockade.

7-[4-(4-Chlorobenzyloxy)benzenesulfonyl]-8-methoxy-3-methyl-2,3,4,5-tetrahydro-1H-3-benzazepinium maleate, Form 2 salt and pharmaceutically acceptable solvates thereof may also exhibit affinity for other receptors not mentioned above, resulting in beneficial antipsychotic activity.

The Form 2 maleate salt of 7-[4-(4-chlorobenzyloxy)benzenesulfonyl]-8-methoxy-3-methyl-2,3,4,5-tetrahydro-1H-3-benzazepine or their pharmaceutically acceptable solvates thereof are of use in the treatment of psychotic disorders.

In a further aspect therefore, the invention provides one or more chemical entities selected from the Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof for use in therapy.

In another aspect, the invention provides one or more chemical entities selected from the Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof for use in the treatment of a condition which requires modulation of a dopamine receptor.

In another aspect, the invention provides one or more chemical entities selected from the Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof for use in the treatment of psychotic disorders.

In another aspect, the invention provides the use of one or more chemical entities selected from the Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof in the manufacture of a medicament for the treatment of a condition which requires modulation of a dopamine receptor.

In another aspect, the invention provides the use of one or more chemical entities selected from the Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof in the manufacture of a medicament for the treatment of psychotic disorders.

In another aspect, the invention provides a method of treating a condition which requires modulation of a dopamine receptor, which comprises administering to a mammal in need thereof an effective amount of one or more chemical entities selected from the Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof.

In another aspect, the invention provides a method of treating psychotic disorders which comprises administering to a mammal in need thereof an effective amount of one or more chemical entities selected from the Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof.

Within the context of the present invention, the terms describing the indications used herein are classified in the Diagnostic and Statistical Manual of Mental Disorders, 4th Edition, published by the American Psychiatric Association (DSM-IV) and/or the International Classification of Diseases, 10th Edition (ICD-10). The various subtypes of the disorders mentioned herein are contemplated as part of the present invention. Numbers in brackets after the listed diseases below refer to the classification code in DSM-IV.

Within the context of the present invention, the term “psychotic disorder” includes:—

Schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

The Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof may also be of use in the treatment of the following disorders:—

Depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90):

Anxiety disorders including Social Anxiety Disorder, Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder and Anxiety Disorder Not Otherwise Specified (300.00):

Substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-induced Persisting Dementia, Alcohol-induced Persisting Amnestic Disorder, Alcohol-induced Psychotic Disorder, Alcohol-induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide:

Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type:

Eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50):

Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23):

Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9):

Enhancement of cognition including the treatment of cognition impairment in other diseases such as schizophrenia, bipolar disorder, depression, other psychiatric disorders and psychotic conditions associated with cognitive impairment, e.g. Alzheimer's disease: and

Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

All of the various forms and sub-forms of the disorders mentioned herein are contemplated as part of the present invention.

“Treatment” includes prophylaxis, where this is appropriate for the relevant condition(s).

It will be appreciated by those skilled in the art that one or more chemical entities selected from the Form 2 maleate salt of 7-[4-(4-chlorobenzyloxy)benzenesulfonyl]-8-methoxy-3-methyl-2,3,4,5-tetrahydro-1H-3-benzazepine and their pharmaceutically acceptable solvates thereof according to the invention may advantageously be used in conjunction with one or more other therapeutic agents, for instance, 5HT₃ antagonists, serotonin agonists, NK-1 antagonists, selective serotonin reuptake inhibitors (SSRI), noradrenaline re-uptake inhibitors (SNRI), non-selective reuptake inhibitors of one or more of serotonin, noradrenaline and norepinephrine, CRF-1 antagonists, tricyclic antidepressants, dopaminergic antidepressants, H₃ antagonists, 5HT_{1A} antagonists, 5HT_{1B} antagonists, 5HT_{1D} antagonists, 5HT₄ partial agonists, D1 agonists, M1 agonists, anticonvulsant agents and/or cyclooxygenase-2 (COX-2) inhibitors.

It will be appreciated that the compounds of the combination or composition may be administered simultaneously (either in the same or different pharmaceutical formulations), separately or sequentially.

Suitable 5HT₃ antagonists which may be used in combination with the Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof include for example one or more chemical entities selected from ondansetron, granisetron and metoclopramide.

Suitable serotonin agonists which may be used in combination with the Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof include for example one or more chemical entities selected from sumatriptan, rauwolscine, yohimbine and metoclopramide.

Suitable SSRIs which may be used in combination with the Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof include for example one or more chemical entities selected from fluoxetine, citalopram, femoxetine, fluvoxamine, paroxetine, indalpine, sertraline and zimeldine.

Suitable SNRIs which may be used in combination with the Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof include for example one or more chemical entities selected from venlafaxine and reboxetine.

Suitable tricyclic antidepressants which may be used in combination with the Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof include for example one or more chemical entities selected from imipramine, amitriptiline, chlomipramine and nortriptiline.

Suitable dopaminergic antidepressants which may be used in combination with the Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof include for example one or more chemical entities selected from bupropion and amineptine.

Suitable anticonvulsant agents which may be used in combination with the Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof include for example one or more chemical entities selected from divalproex, carbamazepine and diazepam.

Suitable NSAID agents which may be used in combination with the Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof include for example one or more chemical entities selected from ibuprofen, aspirin and its active metabolite salicylate.

Suitable COX-2 inhibitors which may be used in combination of the Form 2 maleate salt of 7-[4-(4-chlorobenzyloxy)benzenesulfonyl]-8-methoxy-3-methyl-2,3,4,5-tetrahydro-1H-3-benzazepine and pharmaceutically acceptable solvates thereof include for example rofecoxib (available under the tradename VIOXX®, from Merck, U.S. Pat. No. 5,474,995); celecoxib (available under the tradename CELEBREX®, from Pfizer, U.S. Pat. No. 5,466,823); valdecoxib (available under the tradename BEXTRA®, from Pfizer, U.S. Pat. No. 6,633,272); etoricoxib (available under the tradename ARCOXIA®, from Merck, U.S. Pat. No. 5,861,419); lumiracoxib (available under the tradename PREXIGE®, from Novartis); paracoxib (U.S. Pat. No. 5,932,598); COX-189 from Novartis; BMS347070 from Bristol Myers Squibb; tiracoxib (JTE522) from Japan Tobacco; ABT963 from Abbott; CS502 from Sankyo; 2-(4-ethoxyphenyl)-3-(3-methanesulfonylphenyl)-pyrazolo[1,5-b]pyridazine (GlaxoSmithKline) and 2-butoxy-4-[4-(methylsulfonyl)phenyl]-6-(trifluoromethyl)pyrimidine (GlaxoSmithKline).

The Form 2 maleate salt of 7-[4-(4-chlorobenzyloxy)benzenesulfonyl]-8-methoxy-3-methyl-2,3,4,5-tetrahydro-1H-3-benzazepine and its pharmaceutically acceptable solvates are also suitable for combination with other typical and atypical antipsychotics to provide improved treatment of psychotic disorders. Particular advantages associated with the combinations, uses and methods of treatment of the Form 2 maleate salt of 7-[4-(4-chlorobenzyloxy)benzenesulfonyl]-8-methoxy-3-methyl-2,3,4,5-tetrahydro-1H-3-benzazepine or its pharmaceutically acceptable solvates include equivalent or improved efficacy at doses of administration which are lower than those commonly used for the individual components. Improved treatments of positive symptoms and/or negative symptoms and/or cognitive symptoms of the psychotic disorder may also be observed. The combinations, uses and methods of treatment of the invention may also provide advantages in treatment of patients who fail to respond adequately or who are resistant to treatment with certain antipsychotic agents (also known as neuroleptic agents).

The combination therapies of the invention are preferably administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the Form 2 maleate salt of 7-[4-(4-chlorobenzyloxy)benzenesulfonyl]-8-methoxy-3-methyl-2,3,4,5-tetrahydro-1H-3-benzazepine or its pharmaceutically acceptable solvates and at least one antipsychotic agent are within the scope of the current invention. In one embodiment of adjunctive therapeutic administration as described herein, a patient is typically stabilised on a therapeutic administration of one or more of the components for a period of time and then receives administration of another component. The Form 2 maleate salt of 7-[4-(4-chlorobenzyloxy)benzenesulfonyl]-8-methoxy-3-methyl-2,3,4,5-tetrahydro-1H-3-benzazepine or its pharmaceutically acceptable solvates may be administered as adjunctive therapeutic treatment to patients who are receiving administration of at least one antipsychotic agent, but the scope of the invention also includes the adjunctive therapeutic administration of at least one antipsychotic agent to patients who are receiving administration of the Form 2 maleate salt of the compound of formula (I) or pharmaceutically acceptable solvates thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of the Form 2 maleate salt of 7-[4-(4-chlorobenzyloxy)benzenesulfonyl]-8-methoxy-3-methyl-2,3,4,5-tetrahydro-1H-3-benzazepine or its pharmaceutically acceptable solvates to a patient receiving therapeutic administration of at least one antipsychotic agent. In a further aspect, the invention provides the use of the Form 2 maleate salt of 7-[4-(4-chlorobenzyloxy)benzenesulfonyl]-8-methoxy-3-methyl-2,3,4,5-tetrahydro-1H-3-benzazepine or its pharmaceutically acceptable solvates in the manufacture of a medicament for adjunctive therapeutic administration for the treatment of a psychotic disorder in a patient receiving therapeutic administration of at least one antipsychotic agent. The invention further provides the Form 2 maleate salt of 7-[4-(4-chlorobenzyloxy)benzenesulfonyl]-8-methoxy-3-methyl-2,3,4,5-tetrahydro-1H-3-benzazepine or its pharmaceutically acceptable solvates for use for adjunctive therapeutic administration for the treatment of a psychotic disorder in a patient receiving therapeutic administration of at least one antipsychotic agent.

In a further aspect, the invention provides a method of treatment of a psychotic disorder by adjunctive therapeutic administration of at least one antipsychotic agent to a patient receiving therapeutic administration of the Form 2 maleate salt of 7-[4-(4-chlorobenzyloxy)benzenesulfonyl]-8-methoxy-3-methyl-2,3,4,5-tetrahydro-1H-3-benzazepine or its pharmaceutically acceptable solvates. In a further aspect, the invention provides the use of at least one antipsychotic agent in the manufacture of a medicament for adjunctive therapeutic administration for the treatment of a psychotic disorder in a patient receiving therapeutic administration of the Form 2 maleate salt of 7-[4-(4-chlorobenzyloxy)benzenesulfonyl]-8-methoxy-3-methyl-2,3,4,5-tetrahydro-1H-3-benzazepine or its pharmaceutically acceptable solvates. The invention further provides at least one antipsychotic agent for adjunctive therapeutic administration for the treatment of a psychotic disorder in a patient receiving therapeutic administration of the Form 2 maleate salt of 7-[4-(4-chlorobenzyloxy)benzenesulfonyl]-8-methoxy-3-methyl-2,3,4,5-tetrahydro-1H-3-benzazepine or its pharmaceutically acceptable solvates.

In a further aspect, the invention provides a method of treatment of a psychotic disorder by simultaneous therapeutic administration of the Form 2 maleate salt of 7-[4-(4-chlorobenzyloxy)benzenesulfonyl]-8-methoxy-3-methyl-2,3,4,5-tetrahydro-1H-3-benzazepine or its pharmaceutically acceptable solvates in combination with at least one antipsychotic agent. The invention further provides the use of a combination of the Form 2 maleate salt of 7-[4-(4-chlorobenzyloxy)benzenesulfonyl]-8-methoxy-3-methyl-2,3,4,5-tetrahydro-1H-3-benzazepine or its pharmaceutically acceptable solvates and at least one antipsychotic agent in the manufacture of a medicament for simultaneous therapeutic administration in the treatment of a psychotic disorder. The invention further provides the use of the Form 2 maleate salt of 7-[4-(4-chlorobenzyloxy)benzenesulfonyl]-8-methoxy-3-methyl-2,3,4,5-tetrahydro-1H-3-benzazepine or pharmaceutically acceptable solvates thereof in the manufacture of a medicament for simultaneous therapeutic administration with at least one antipsychotic agent in the treatment of a psychotic disorder. The invention further provides the Form 2 maleate salt of 7-[4-(4-chlorobenzyloxy)benzenesulfonyl]-8-methoxy-3-methyl-2,3,4,5-tetrahydro-1H-3-benzazepine or pharmaceutically acceptable solvates thereof for use for simultaneous therapeutic administration with at least one antipsychotic agent in the treatment of a psychotic disorder. The invention further provides the use of at least one antipsychotic agent in the manufacture of a medicament for simultaneous therapeutic administration with the Form 2 maleate salt of 7-[4-(4-chlorobenzyloxy)benzenesulfonyl]-8-methoxy-3-methyl-2,3,4,5-tetrahydro-1H-3-benzazepine or pharmaceutically acceptable solvates thereof in the treatment of a psychotic disorder.

In further aspects, the invention provides a method of treatment of a psychotic disorder by simultaneous therapeutic administration of a pharmaceutical composition comprising the Form 2 maleate salt of 7-[4-(4-chlorobenzyloxy)benzenesulfonyl]-8-methoxy-3-methyl-2,3,4,5-tetrahydro-1H-3-benzazepine or pharmaceutically acceptable solvates thereof and at least one mood stabilising or antimanic agent, a pharmaceutical composition comprising the Form 2 maleate salt of 7-[4-(4-chlorobenzyloxy)benzenesulfonyl]-8-methoxy-3-methyl-2,3,4,5-tetrahydro-1H-3-benzazepine or pharmaceutically acceptable solvates thereof and at least one mood stabilising or antimanic agent, the use of a pharmaceutical composition comprising the Form 2 maleate salt of 7-[4-(4-chlorobenzyloxy)benzenesulfonyl]-8-methoxy-3-methyl-2,3,4,5-tetrahydro-1H-3-benzazepine or pharmaceutically acceptable solvates thereof and at least one mood stabilising or antimanic agent in the manufacture of a medicament for the treatment of a psychotic disorder, and a pharmaceutical composition comprising the Form 2 maleate salt of 7-[4-(4-chlorobenzyloxy)benzenesulfonyl]-8-methoxy-3-methyl-2,3,4,5-tetrahydro-1H-3-benzazepine or pharmaceutically acceptable solvates thereof and at least one mood stabilising or antimanic agent for use in the treatment of a psychotic disorder.

In a further aspect, the invention provides a kit-of-parts for use in the treatment of a psychotic disorder comprising a first dosage form comprising the Form 2 maleate salt of 7-[4-(4-chlorobenzyloxy)benzenesulfonyl]-8-methoxy-3-methyl-2,3,4,5-tetrahydro-1H-3-benzazepine or pharmaceutically acceptable solvates thereof and one or more further dosage forms each comprising an antipsychotic agent for simultaneous therapeutic administration.

Examples of antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benzisothiazolyl-piperazines; triazine such as lamotrigine; dibenzoxazepines, such as loxapine; dihydroindolones, such as molindone; aripiprazole; and derivatives thereof that have antipsychotic activity.

Examples of tradenames and suppliers of selected antipsychotic drugs that are suitable for use in the present invention are as follows: clozapine (available under the tradename CLOZARIL®, from Mylan, Zenith Goldline, UDL, Novartis); olanzapine (available under the tradename ZYPREXA®, from Lilly; ziprasidone (available under the tradename GEODON®, from Pfizer); risperidone (available under the tradename RISPERDAL®, from Janssen); quetiapine fumarate (available under the tradename SEROQUEL®, from AstraZeneca); sertindole (available under the tradename SERLECT®); amisulpride (available under the tradename SOLION®, from Sanofi-Synthelabo); haloperidol (available under the tradename HALDOL®, from Ortho-McNeil); haloperidol decanoate (available under the tradename HALDOL Decanoate®); haloperidol lactate (available under the tradenames HALDOL® and INTENSOL®) chlorpromazine (available under the tradename THORAZINE®, from SmithKline Beecham (GSK); fluphenazine (available under the tradename PROLIXIN®, from Apothecon, Copley, Schering, Teva, and American Pharmaceutical Partners, Pasadena); fluphenazine decanoate (available under the tradename PROLIXIN Decanoate®); fluphenazine enanthate (available under the tradename PROLIXIN®); fluphenazine hydrochloride (available under the tradename PROLIXIN®); thiothixene (available under the tradename NAVANE®; from Pfizer); thiothixene hydrochloride (available under the tradename NAVANE®); trifluoperazine (10-[3-(4-methyl-1-piperazinyl)propyl]-2-(trifluoromethyl)phenothiazine dihydrochloride, available under the tradename STELAZINE®, from SmithKlien Beckman; perphenazine (available under the tradename TRILAFON®; from Schering); perphenazine and amitriptyline hydrochloride (available under the tradename ETRAFON TRILAFON®); thioridazine (available under the tradename MELLARIL®; from Novartis, Roxane, HiTech, Teva, and Alpharma); molindone (available under the tradename MOBAN®, from Endo); molindone hydrochloride (available under the tradename MOBAN®); loxapine (available under the tradename LOXITANE®; from Watson); loxapine hydrochloride (available under the tradename LOXITANE®); and loxapine succinate (available under the tradename LOXITANE®). Furthermore, benperidol (Glianimon®), perazine (Taxilan®) or melperone (Eunerpan®)) may be used.

Other suitable antipsychotic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®), prochlorperazine (available under the tradename COMPAZINE®), methotrimeprazine (available under the tradename NOZINAN®), pipotiazine (available under the tradename PIPOTRIL®), iloperidone, pimozide and flupenthixol.

In one further aspect of the invention, suitable antipsychotic agents include olanzapine, risperidone, quetiapine, aripiprazole, haloperidol, clozapine, ziprasidone and osanetant.

For use in medicine, 7-[4-(4-chlorobenzyloxy)benzenesulfonyl]-8-methoxy-3-methyl-2,3,4,5-tetrahydro-1H-3-benzazepinium maleate, Form 2 or a pharmaceutically acceptable solvate thereof are usually administered as a standard pharmaceutical composition. The pharmaceutical composition can be for use in the treatment of any of the conditions described herein.

Therefore in a further aspect of the present invention there is provided a pharmaceutical composition comprising one or more chemical entities selected from 7-[4-(4-chlorobenzyloxy)benzenesulfonyl]-8-methoxy-3-methyl-2,3,4,5-tetrahydro-1H-3-benzazepinium maleate, Form 2 and a pharmaceutically acceptable solvate thereof, together with a pharmaceutically acceptable carrier.

7-[4-(4-Chlorobenzyloxy)benzenesulfonyl]-8-methoxy-3-methyl-2,3,4,5-tetrahydro-1H-3-benzazepinium maleate, Form 2 or a pharmaceutically acceptable solvate thereof may be administered by any convenient method, for example by oral, parenteral (e.g. intravenous), buccal, sublingual, nasal, rectal or transdermal administration and the pharmaceutical compositions adapted accordingly.

7-[4-(4-Chlorobenzyloxy)benzenesulfonyl]-8-methoxy-3-methyl-2,3,4,5-tetrahydro-1H-3-benzazepinium maleate, Form 2 or a pharmaceutically acceptable solvate thereof can be formulated as liquids or solids, for example syrups, suspensions or emulsions, tablets, capsules and lozenges.

A liquid formulation will generally consist of a suspension or solution of 7-[4-(4-chlorobenzyloxy)benzenesulfonyl]-8-methoxy-3-methyl-2,3,4,5-tetrahydro-1H-3-benzazepinium maleate, Form 2 or a pharmaceutically acceptable solvate thereof in a suitable liquid carrier(s) for example an aqueous solvent such as water, ethanol or glycerine, or a non-aqueous solvent, such as polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and cellulose.

A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of 7-[4-(4-chlorobenzyloxy)benzenesulfonyl]-8-methoxy-3-methyl-2,3,4,5-tetrahydro-1H-3-benzazepinium maleate, Form 2 or a pharmaceutically acceptable solvate thereof in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active substance in a pharmaceutically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a propellant which can be a compressed gas such as compressed air or an organic propellant such as a fluorochloro-hydrocarbon. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches. The composition is suitably in unit dose form such as a tablet, capsule or ampoule.

7-[4-(4-Chlorobenzyloxy)benzenesulfonyl]-8-methoxy-3-methyl-2,3,4,5-tetrahydro-1H-3-benzazepinium maleate, Form 2 or a pharmaceutically acceptable solvate thereof will normally be administered in a daily dosage regimen (for an adult patient) of, for example, an oral dose of between 1 mg and 250 mg, such as between 1 mg and 250 mg, such as between 2 mg and 120 mg, e.g. between 2 and 50 mg or an intravenous, subcutaneous, or intramuscular dose of between 0.1 mg and 120 mg, for example between 0.1 mg and 50 mg, e.g. between 1 and 25 mg of 7-[4-(4-chlorobenzyloxy)benzenesulfonyl]-8-methoxy-3-methyl-2,3,4,5-tetrahydro-1H-3-benzazepinium calculated as the free base, the compound being administered 1 to 4 times per day. Suitably the compounds will be administered for a period of continuous therapy, for example for a week or more.

No adverse toxicological effects have been observed for compounds of the invention at doses expected to be approved for therapeutic administration.

The invention is further illustrated by the following non-limiting examples:

### Description 1

### Preparation of 7-[4-(4-chlorobenzyloxy)benzenesulfonyl]-8-methoxy-3-methyl-2,3,4,5-tetrahydro-1H-3-benzazepinium Maleate

### Anhydrate (D1)

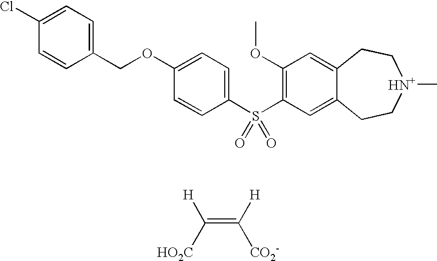

A solution of maleic acid (0.204 g, 1.76 mmol) in ethanol (2 mL) was added to a boiling solution of 7-[4-(4-chlorobenzyloxy)-benzenesulfonyl]-8-methoxy-3-methyl-2,3,4,5-tetrahydro-1H-3-benzazepine (0.75 g, 1.6 mmol) in ethanol (5.5 mL) and the resulting solution heated under reflux for 30 minutes. The solution was cooled to ambient temperature and stirred for 1 hour, resulting in some oiling of the product. Stirring was stopped and the mixture left to stand overnight resulting in a two phase mixture. The mixture was stirred and heated to reflux; a homogeneous solution was briefly obtained when the heating bath was 55° C., then the product crystallized. Heating was continued to reflux, the slurry stirred for 30 minutes, then cooled to ambient temperature, stirred for 30 minutes, then cooled to 0-5° C. and stirred for a further 30 minutes. The resulting slurry was filtered; the cake was washed with cold ethanol (3.5 mL) and dried under vacuum at 50° C. to yield the title product (D1) as a white solid (0.86 g).

### Description 2

### Preparation of 7-[4-(4-chlorobenzyloxy)benzenesulfonyl]-8-methoxy-3-methyl-2,3,4,5-tetrahydro-1H-3-benzazepinium Maleate

### Anhydrate (D2)

A solution of maleic acid (27.1 g, 233.4 mmol) in ethanol (100 mL) was added portionwise to a boiling solution of 7-[4-(4-chlorobenzyloxy)-benzenesulfonyl]-8-methoxy-3-methyl-2,3,4,5-tetrahydro-1H-3-benzazepine (100.1 g, 212.0 mmol) in ethanol (1.05 L) and the resulting solution allowed to stir for 10 minutes and return to reflux. The solution was cooled to 75° C., seeded with 7-[4-(4-chlorobenzyloxy)benzenesulfonyl]-8-methoxy-3-methyl-2,3,4,5-tetrahydro-1H-3-benzazepinium maleate, Anhydrate (100.8 mg) then cooled to ambient temperature. The resulting slurry was stirred at ambient temperature for 2 hours and filtered; the cake was washed with ethanol (300 mL) and dried under vacuum at 60° C. to yield the title product (D2) as a white solid (122.4 g). Onset of melt 170-172° C.; δ_{H} (400 MHz, DMSO) 2.81 (3H, s, NCH₃), 3.10 (4H, br s, CH₂CH₂), 3.34 (4H, br s, CH₂CH₂), 3.72 (3H, s, OCH₃), 5.18 (2H, s, ArCH₂), 6.02 (2H, s, —CH═CH—), 7.07 (1H, s, ArH), 7.17 (2H, d, J=7, ArH), 7.46 (4H, m, ArH), 7.80 (2H, d, J=7, ArH), 7.82 (1H, s, ArH), 9.0-10.0 (1H, br s); MS (ES+) m/z 474 (MH⁺), 472 (MH⁺, 100%) 192.

### EXAMPLE 1

### 7-[4-(4-Chlorobenzyloxy)benzenesulfonyl]-8-methoxy-3-methyl-2,3,4,5-tetrahydro-1H-3-benzazepinium Maleate

### Anhydrate Form 2 (E1)

7-[4-(4-Chlorobenzyloxy)benzenesulfonyl]-8-methoxy-3-methyl-2,3,4,5-tetrahydro-1H-3-benzazepinium maleate, Anhydrate prepared according to description 2 above (1 g) was slurried in 2-butanone and the slurry heated from ambient to 40° C., then cooled to 0° C. and reheated to 40° C. at 0.3° C./minute using a Metz Syn¹⁰ Reaction Station, supplied by Radleys Discovery Technologies. The temperature cycle was run 10 times, then the slurry cooled to 20° C. and held for 10 hours. The slurry was filtered and the resulting solid dried under vacuum to give the title product (E1).

### EXAMPLE 2

### Preparation of 7-[4-(4-chlorobenzyloxy)benzenesulfonyl]-8-methoxy-3-methyl-2,3,4,5-tetrahydro-1H-3-benzazepinium Maleate

### Anhydrate Form 2 (E2)

7-[4-(4-Chlorobenzyloxy)benzenesulfonyl]-8-methoxy-3-methyl-2,3,4,5-tetrahydro-1H-3-benzazepinium maleate, Anhydrate prepared according to description 2 above (25 g) was dissolved in 9:1 acetonitrile:water (60 mL) at 60° C., stirred for 1 hour, cooled to 50° C. and seeded using a seed of 7-[4-(4-chlorobenzyloxy)benzenesulfonyl]-8-methoxy-3-methyl-2,3,4,5-tetrahydro-1H-3-benzazepinium maleate, Anhydrate Form 2 (0.25 g) as may be prepared for example using the method of Example 1 above. The resulting mixture was cooled to 15° C. over 12 hours and stirred at 15° C. for a further 8 hours. The solid was filtered, washed with 9:1 acetonitrile:water (10 mL) and dried under vacuum at 40° C. to give the title product (E2) as a white solid (18.79 g). A sample was further dried under vacuum at 100° C. for 6 hours. Onset of melt 171-172° C.; δ_{H} (400 MHz, DMSO) 2.81 (3H, s, NCH₃), 3.10 (4H, br s, CH₂CH₂), 3.31 (4H, br s, CH₂CH₂), 3.72 (3H, s, OCH₃), 5.18 (2H, s, ArCH₂), 6.02 (2H, s, —CH═CH—), 7.07 (1H, s, ArH), 7.17 (2H, d, J=7, ArH), 7.45 (4H, m, ArH), 7.80 (2H, d, J=7, ArH), 7.82 (1H, s, ArH), 9.5-10.0 (1H, br s); MS (ES+) m/z 474 (MH⁺), 472 (MH⁺, 100%) 192.

### EXAMPLE 3

### Preparation of 7-[4-(4-chlorobenzyloxy)benzenesulfonyl]-8-methoxy-3-methyl-2,3,4,5-tetrahydro-1H-3-benzazepinium Maleate

### Form 2 Tetrahydrofuran Solvate (E3)

7-[4-(4-Chlorobenzyloxy)benzenesulfonyl]-8-methoxy-3-methyl-2,3,4,5-tetrahydro-1H-3-benzazepinium maleate, Anhydrate Form 2 (2.5 g) was dissolved in tetrahydrofuran (250 mL) at reflux, stirred for 30 minutes, cooled to 50° C. and seeded using a seed of 7-[4-(4-chlorobenzyloxy)benzenesulfonyl]-8-methoxy-3-methyl-2,3,4,5-tetrahydro-1H-3-benzazepinium maleate, Anhydrate Form 2 (0.025 g). The resulting mixture was cooled to ambient temperature over 1 hour and stirred at ambient temperature for 141.75 hours. The solid was filtered, washed with tetrahydrofuran (25 mL) and dried under vacuum at 50° C. for 18 hours to give the title product (E3) as a white solid (1.16 g). Onset of melt 171-172° C.; tetrahydrofuran by GC Analysis 2.4%; δ_{H} (400 MHz, DMSO) 1.78 (4H, m, CH₂CH₂), 2.83 (3H, s, NCH₃), 3.12 (4H, br s, CH₂CH₂), 3.33 (4H, br s, CH₂CH₂), 3.62 (4H, m, CH₂OCH₂), 3.74 (3H, s, OCH₃), 5.21 (2H, s, ArCH₂), 6.04 (2H, s, —CH═CH—), 7.10 (1H, s, ArH), 7.19 (2H, d, J=9, ArH), 7.48 (4H, m, ArH), 7.83 (2H, d, J=9, ArH), 7.84 (1H, s, ArH), 9.5-10.0 (1H, br s); MS (ES+) m/z 474 (MH⁺), 472 (MH⁺, 100%) 192.

TABLE 1
XRPD angles and d spacings for 7-[4-(4-chlorobenzyloxy)benzene-
sulfonyl]-8-methoxy-3-methyl-2,3,4,5-tetrahydro-1H-3-
benzazepinium maleate, Anhydrate Form 2

| Pos. [°2Th.] | d-spacing[Å] |
|---|---|
| 5.6 | 15.8 |
| 11.1 | 7.9 |
| 13.3 | 6.7 |
| 14.0 | 6.3 |
| 14.5 | 6.1 |
| 16.5 | 5.4 |
| 16.7 | 5.3 |
| 17.4 | 5.1 |
| 18.6 | 4.8 |
| 19.5 | 4.5 |
| 20.5 | 4.3 |
| 20.6 | 4.3 |
| 20.9 | 4.2 |
| 21.1 | 4.2 |
| 22.3 | 4.0 |
| 22.7 | 3.9 |
| 23.4 | 3.8 |
| 23.6 | 3.8 |
| 23.8 | 3.7 |
| 25.4 | 3.5 |
| 26.7 | 3.3 |
| 27.5 | 3.2 |
| 27.9 | 3.2 |
| 29.2 | 3.1 |

TABLE 2
XRPD angles and d spacings for 7-[4-(4-chlorobenzyloxy)benzene-
sulfonyl]-8-methoxy-3-methyl-2,3,4,5-tetrahydro-1H-3-
benzazepinium maleate, Form 2 tetrahydrofuran solvate

| Pos. [°2Th.] | d-spacing[Å] |
|---|---|
| 5.6 | 15.8 |
| 13.2 | 6.7 |
| 14.3 | 6.2 |
| 14.8 | 6.0 |
| 16.1 | 5.5 |
| 16.5 | 5.4 |
| 16.7 | 5.3 |
| 18.6 | 4.8 |
| 19.4 | 4.6 |
| 20.2 | 4.4 |
| 20.4 | 4.4 |
| 20.7 | 4.3 |
| 21.3 | 4.2 |
| 22.4 | 4.0 |
| 23.4 | 3.8 |
| 23.5 | 3.8 |
| 23.8 | 3.7 |
| 25.1 | 3.5 |
| 26.6 | 3.4 |
| 27.6 | 3.2 |
| 28.0 | 3.2 |
| 29.2 | 3.1 |
| 32.2 | 2.8 |
| 33.0 | 2.7 |
| 34.8 | 2.6 |

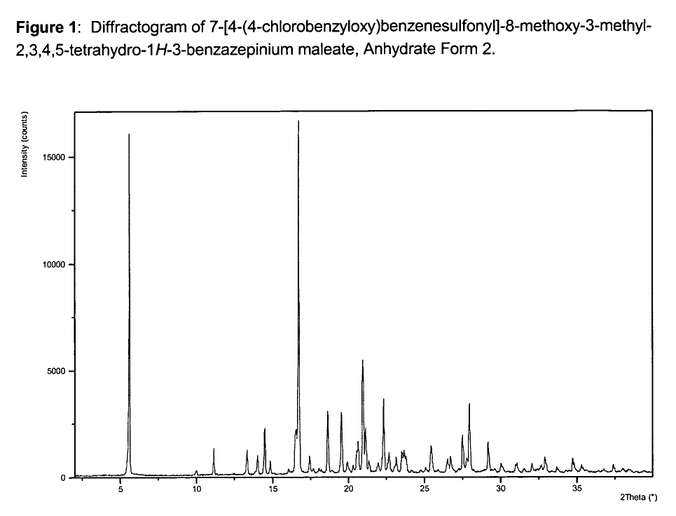

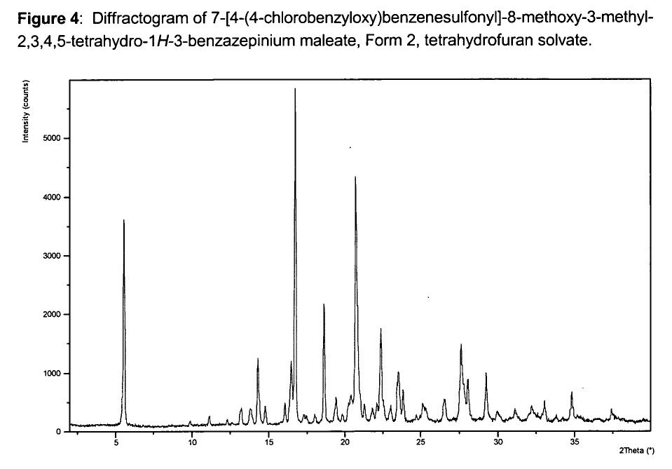

The X-Ray Powder Diffraction (XRPD) analysis shown in FIGS. 1 and 4 was performed on a Phillips X'pert Pro powder diffractometer, Model PW3040/60, serial number DY1379 using an X'Celerator detector. The acquisition conditions were; radiation: Cu K_{α}, generator tension: 40 kV, generator current: 45 mA, start angle: 2.0°2θ, end angle: 40.0°2θ, step size: 0.0167°2θ, time per step: 31.75 seconds. The sample was prepared using silicon wafer technique. Peaks with relative intensities greater than 5% are included in Tables 1 and 2.

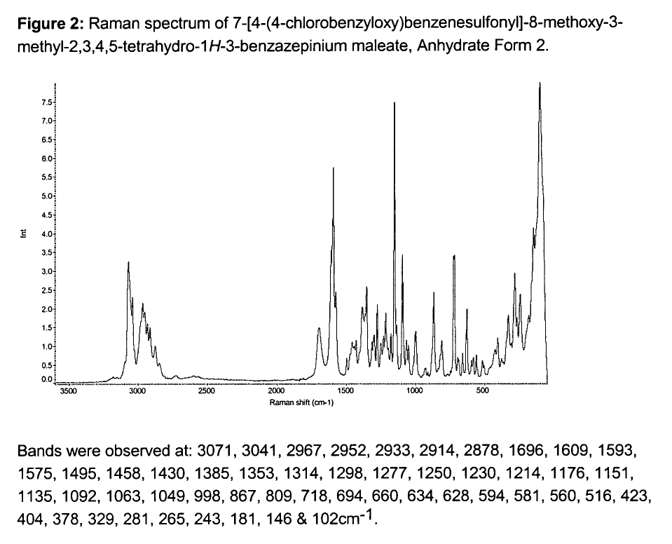

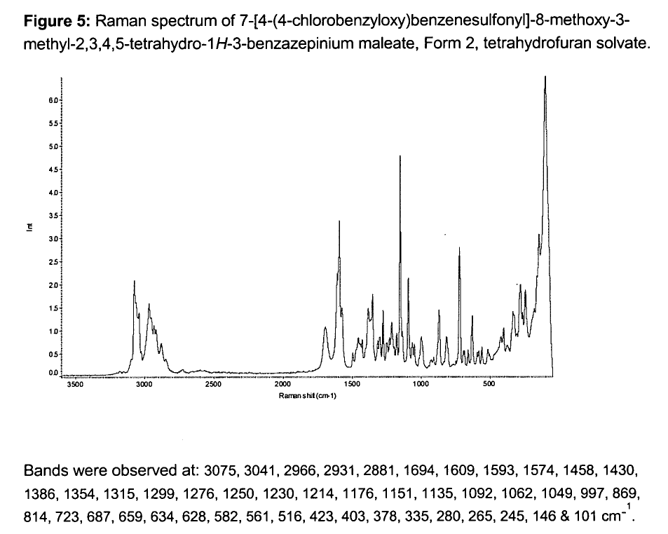

The Raman spectrum of the products (FIGS. 2 and 5) was recorded with the sample in a NMR tube using a Nicolet 960 E.S.P. FT-Raman spectrometer, serial number 5880, at 4 cm⁻¹ resolution with excitation from a Nd:VO₄ laser (1064 nm) with a power output of 400 mW. An absolute threshold of 0.5 and sensitivity of 65% were applied for the purpose of peak selection.

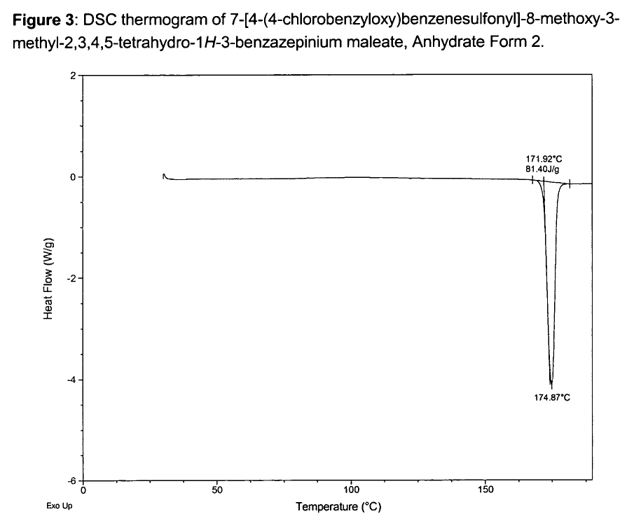

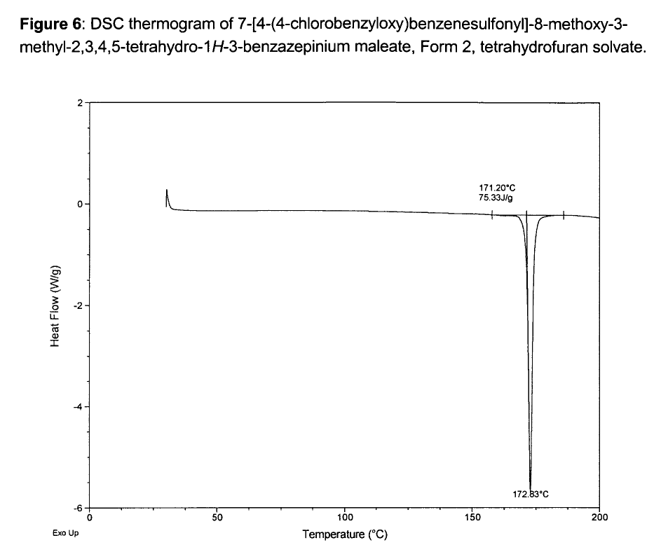

The DSC thermogram of the products (FIGS. 3 and 6) was recorded on a Thermal Analysis DSC Q1000, serial number 1000-0060. The sample was heated at 10° C. min⁻¹ in an open pan.

All publications, including but not limited to patents and patent applications, cited in this specification are herein incorporated by reference as if each individual publication were specifically and individually indicated to be incorporated by reference herein as though fully set forth.

1. One or more chemical entities selected from a crystalline form of 7-[4-(4-chlorobenzyloxy)benzenesulfonyl]-8-methoxy-3-methyl-2,3,4,5-tetrahydro-1H-3-benzazepinium maleate, Form 2 and a pharmaceutically acceptable solvate thereof.

2. 7-[4-(4-Chlorobenzyloxy)benzenesulfonyl]-8-methoxy-3-methyl-2,3,4,5-tetrahydro-1H-3-benzazepinium maleate, Anhydrate Form 2 characterised by its X-Ray powder diffraction (XRPD) pattern with signals substantially as listed in Table 1.

3. 7-[4-(4-Chlorobenzyloxy)benzenesulfonyl]-8-methoxy-3-methyl-2,3,4,5-tetrahydro-1H-3-benzazepinium maleate, Form 2, tetrahydrofuran solvate characterised by its X-Ray powder diffraction (XRPD) pattern with signals substantially as listed in Table 2.

4. A pharmaceutical composition comprising a chemical entity as claimed in claim 1 and a pharmaceutically acceptable carrier.

5. (canceled)

6. A chemical entity according to claim 1 for use in the treatment of a psychotic disorder.

7. (canceled)

8. A method of treatment of a psychotic disorder in mammals including humans, which comprises administering to a mammal in need thereof an effective amount of a chemical entity according to claim 1.
